(19) United States
(12) Patent Application Publication
(10) Pub. No.: US 2007/0299539 A1
(43) Pub. Date: Dec. 27, 2007

(54) ULTRASOUND ACCELERATED TISSUE ENGINEERING PROCESS

(76) Inventors: Shadi Othman, Chicago, IL (US); Nadia Halim, Chicago, IL (US); Richard L. Magin, Western Springs, IL (US); Jessy Mouannes, Elmwood Park, IL (US); Neelima Vidula, Naperville, IL (US)

Correspondence Address: GREENLEE WINNER AND SULLIVAN P C, 4875 PEARL EAST CIRCLE, SUITE 200, BOULDER, CO, 80301, US

(21) Appl. No.: 11/734,612

(22) Filed: Apr. 12, 2007

Related U.S. Application Data
(60) Provisional application No. 60/791,632, filed on Apr. 12, 2006.

(51) Int. Cl. A61F 2/02; C12N 13/00

(52) U.S. Cl. 623/23.72; 435/173.1; 435/173.8

(57) ABSTRACT

In an aspect the invention is a method of preparing a cell or tissue implant for insertion into a patient in need of treatment by obtaining a transplantable cell population, culturing the cell population in a culture media and exposing the cell population to a sonic or ultrasonic stimulation, wherein the stimulation provides a capability for an enhanced implant outcome parameter. The method provides enhanced autologous bone implant procedures by reducing the time required for a patient's own cells to sufficiently undergo osteogenesis, thereby reducing the waiting time for an autologous bone implant. The extent of osteogenesis is optionally monitored non-invasively by magnetic resonance spectroscopy.

### CROSS-REFERENCE TO RELATED APPLICATIONS

This application claims the benefit of U.S. Provisional Application No. 60/791,632, filed Apr. 12, 2006.

### BACKGROUND OF THE INVENTION

Bone loss is a significant medical issue in that millions of individuals currently experience bone loss (Xu et al, 2005). In the United States alone, an estimated 500,000 annual surgeries involving bone restorative substitutes are performed, with more than 15 billion dollars spent each year in treating bone conditions. These statistics indicate that the market for processes that provide a solution to the problem of bone loss is tremendous. Thus, implants developed via enhanced bone tissue engineering processes that improve these medical conditions are of interest to healthcare professionals, patients, and those involved in industry, among others.

Implantation of bone-replacing constructs and tissue is an active area or research because the repercussions associated with bone loss are severe. In particular, it is important to replace lost bone in patients suffering a bone defect. Present methods for specific-site structural and functional bone defect repair are autologous bone grafts (Mauney et al, 2005) or autografts. Autografts do not present an immune rejection problem because the bone tissue is transplanted into the patient from another region of the patient's own body (Rahaman et al, 2005). Autrografts do, however, present certain complications including significant donor site morbidity (death of tissue remaining in the region from which the donor tissue was removed), infection, malformation, and subsequent loss of graft function (Mauney et al, 2005).

An alternative therapy involves transplantation of allograft bone or bone tissue from a donor. (Mauney et al, 2005). Although allograft bone is effective in treating bone loss, there are several problems associated with that therapy. First, a compatible donor must be found (Jones et al, 2006) in order to minimize the possibility of immune rejection by the patient; second, there is a risk of potential disease transmission from the donor to the patient; third, donor site morbidity can occur (Jones et al, 2006); and, finally, there is a limited supply of donor tissue (Mauney et al, 2005). Therefore, patients often experience long waiting periods before receiving the transplant, due to the scarcity of tissue donors, and this can exacerbate bone tissue loss (Jones et al, 2006).

Because of the limitations associated with autologous transplantation and allograft transplantation, much effort is directed in the field of bone tissue engineering. Implants developed via tissue engineering applications may be a more viable solution to the problem of bone loss than conventional solutions. In contrast with bone graft transplants, tissue-engineered implants are not subject to patient-donor tissue biocompatibility issues, because donor tissue is unnecessary. Also, morbidity of the site of extracted tissue is not a problem, since implants can be developed by obtaining stem cells from the patient, thereby avoiding the morbidity issue. Additionally, implants are generally more readily available to patients than transplants, which reduce the time for initiation of bone loss treatment (Jones et al, 2006). Therefore, bone tissue engineering may become the standard treatment for bone loss.

A significant obstacle to bone tissue engineering using a patient's own cells is the length of time required to obtain a construct suitable for implantation into the patient. Accordingly, reducing the culture time of stem cells is necessary to increase the effectiveness of tissue engineered implants. A potential method for reducing the required culture time of stem cells to generate a tissue-engineered bone implant involves mechanical stimulation. For example, Aaron et al (2004) demonstrate that electric and electromagnetic fields can accelerate bone formation (osteogenesis) and healing, particularly in osteotomies and spine fusions, both in vivo and in vitro.

“Electrical properties of bone” (Lakes 2005) describes another type of stimulation that can be achieved by means of a piezoelectric actuator. It has been demonstrated by several researchers that bone is piezoelectric. The piezoelectric nature of bone indicates that any mechanical stress applied to bone can produce an electric polarization of the tissue, and any electric field applied to bone can cause mechanical strain of the tissue. Piezoelectric effects occur in the kilo-hertz range, well above that of physiologically significant frequencies.

Pilla (2002) describes how low-intensity electromagnetic and mechanical modulations of bone growth and repair are equivalent. There is a time-varying electric field, E(t), associated with both types of stimuli, which serves as a main messenger regulating cellular activity, therefore acting as a growth stimulus. This electric field can be either directly induced with electric and electromagnetic devices, or indirectly induced by an applied mechanical stress. One way to generate the latter is to use a piezoelectric actuator as described previously, and another way, which has also been demonstrated to work effectively for both in vivo and in vitro bone formation and repair by Nolte et al. (2001), is to use low-intensity ultrasound waves. Bone repair is significantly enhanced by both electromagnetic fields and ultrasound (US) signals (Pilla et al, 2002).

Studies indicate that ultrasound therapy may enhance the biological repair process in vivo. For example, Klug et al. accelerated the healing of closed lower-extremity fractures in rabbits by 18%. Pilla et al. showed mid-shift tibial osteotomies in rabbits accelerated the recovery of torsioinal strength and stiffness, with a recovery to intact fibul occurring by the seventeenth day compared to 28 days for control limbs. Furthermore, US-treated injured bones achieved biomechanical integrity in about half the time of treated bones. US treatment can increase bone strength at the fracture site (Wang et al.; showing maximum torque to failure of the US-treated femora 22% greater than control). Bone stiffness increased with higher-frequency US bursts (0.5 MHz v. 1.5 MHz burst). Other properties that have been suggested to improve with US-treatment of bone include bone-mineral content, bone-mineral density, peak torque, and peak stiffness (Jingushi et al.). Studies suggest that a pulse width of 200-μs and a 1-kHz repetition rate are reflective of optimal US parameters for fracture healing.

In vitro studies provide additional insight into the biological effect of US on tissue. In response to a variety of US signal intensity therapy, a number of different cells are shown to change influx/efflux rate of potassium ions, change cell metabolism including increasing calcium incorporation), change enzyme activity and/or growth factor expression or level. Furthermore, US-treatment of cultured chondrocytes appears to upregulate aggrecan gene expression, a gene involved in the fracture-healing process.

From the forgoing, it is apparent that although US-treatment is established as a therapy for improving tissue healing in vivo, there remains a need for US-processes to accelerate cell growth in the context of tissue engineering. In contrast to those studies that improve bone fracture healing, an aspect of the present invention relates to accelerating in vitro growth of cells to decrease the time required to obtain a suitable bone implant from those in vitro cells. This acceleration in growth and development provides an ability to generate autologous tissue engineered implants, wherein the time for which a patient awaits implantation is minimized.

### SUMMARY OF THE INVENTION

The methods disclosed herein rely on stimulation of cells by sonic or ultrasonic-generated forces to enhance mechanical signal transduction, thereby improving growth and development of the cells. In particular, ultrasonic stimulation of cells is useful in situations where it is desirable to maximize cell growth and development, such as for autologous implantation, where reducing the time for suitable implant generation results in increased cost savings and improved patient care.

An aspect of the invention provides methods and related devices for preparing a cell or tissue implant by sonic or ultrasonic stimulation, to enhance an implant outcome parameter by exposing a transplantable cell population to the stimulation. The stimulation may be applied prior to, after or prior to and after the implant is inserted into the patient. The cells are optionally cultured for a culturing time. This culturing time is any time length sufficient to enhance one or more implant outcome parameters by providing sufficient time for in vitro sonic or ultrasonic stimulation and associated cellular responses thereto.

Depending on the particular cell population, the implant optionally comprises a biocompatible scaffold to which the cell population is introduced. In this aspect, the sonic or ultrasonic stimulation is applied prior to, after, or prior to and after the cells are introduced to the scaffold. In an embodiment the method is ultrasound or sonic stimulation of a cell population introduced to a biocompatible scaffold, for example a mesenchymal cell population or a bone cell derived therefrom on a collagen scaffold. In an aspect, a mesenchymal cell population is exposed to a signal that results in differentiation of at least a portion of the mesenchymal cell population to a bone-producing cell population (e.g., osteoblasts)

In an aspect, the implant outcome parameter that is enhanced by the sonic or ultrasonic stimulation is one or more of accelerated cell growth or proliferation, reduced time for implant generation, increased mineral deposition, increased osteogenesis, or any other measurable parameter indicative of an implant improved by the sound or ultrasound stimulation. In aspects where cells are introduced to a scaffold, another potential indication of accelerated cell growth is an increased rate of scaffold break-down by the cells. This is advantageous as potential host immune response is further minimized by the absorption of the scaffold by the surrounding cells so that only the cell population and related extracellular matrix remains.

In an embodiment, the implant is an autologous implant where the cell population is obtained from the patient in need of treatment. Alternatively, the cell population is obtained from a donor of the same species of the patient, or of a different species. In an aspect, the cell population is expanded by cell culturing methods known in the art prior to introducing the cell population to a biocompatible scaffold and/or prior to implantation in the patient. For example, expansion can include growing the cell population to ensure that a sufficient number of cells are generated so that they may be applied (either to the implant or to the patient), at a cell concentration selected from a range of between 1×10⁶ to 1×10⁷ cells/mL.

In an aspect, the cell population comprises mesenchymal stem cells, such as mesenchymal stem cells obtained from a patient's bone marrow. Alternatively, the cell population is a tissue-specific cell such as a bone cell, cartilage cell, or other tissue-specific cell capable of responding to physical forces generated by low-intensity ultrasound. A stem cell population that is a precursor of the tissue in which the implant is to be implanted is optionally exposed to a differentiating signal such that at least a portion of the (mesenchymal) stem cells differentiate into osteoblast cells prior, during, or prior and during said ultrasonic stimulation, and optionally before or after introduction of the cells to a biocompatible scaffold.

In an aspect, the sonic or ultrasonic stimulation of the invention has a number of physical parameters that describe the stimulation in a quantitative manner, such as one or more parameters selected from the group consisting of intensity, operating frequency, pulse width, pulse repetition rate, individual treatment duration and overall treatment duration. For example, in an aspect the stimulation is a low-intensity ultrasound stimulation having an intensity of between about 10 and 60 mW/cm², or about 30 mW/cm²; an operating frequency between about 10 Hz and 10 MHz, or of about 1.5 MHz; a pulse width between about 50 μsec and 500 μsec, or about 200 μsec; and a pulse repetition rate between about 500 Hz and 5 kHz for the US stimulation, or about 1 kHz. In an aspect the stimulation is an ultrasonic or sonic stimulation having an operating frequency of between 10 Hz and 10 MHz and a pulse repetition rate selected from between 500 Hz and 5 kHz. Methods may use a stimulus having any one or more of these parameters having any value, so long as there is an enhanced implant parameter outcome that is measurably detectable.

In an aspect, the stimulation is applied intermittently. In an aspect, the intermittent application has substantial periodicity. Substantial periodicity refers to a relatively constant time between stimulation application, but that individual applications may be omitted. For example, with a daily application of ultrasound stimulation, the weekends are optionally skipped so that the application is said to be “substantially periodic.” In an aspect, an individual treatment has a daily treatment duration selected from a range between 10 minutes and 30 minutes, or about 20 minutes a day. In an aspect, the total number of stimulations is from between 5 and 28, 5 and 21, 10 and 16, or any subcombination thereof. In an aspect, the stimulation occurs daily. In an aspect, the stimulation occurs two times a day, or two or more times a day and repeated for between about 5 days and 21 days.

Any of the implants generated by the methods of the invention are optionally implanted into a patient.

In an embodiment, the implant is a bone implant and the outcome parameter is accelerated osteogenesis. Accelerated osteognesis results in a decreased time required for bone implant generation compared to a bone implant not exposed to the sonic or ultrasonic stimulation. In particular, the stimulation decreases the time required to reach a certain level of cellular growth or proliferation, such as a decrease in time to implant by about 20%, 25%, 50%, or better, relative to a bone implant not stimulated with a sonic or ultrasonic stimulation.

For quality monitoring, any of the methods optionally includes a means for assessing an implant outcome parameter. The monitoring is preferably non-invasive such as by magnetic resonance microscopy (MRM) that measures osteogenesis (e.g., extent of mineralization). In an embodiment, the invention provides a magnetic resonance spectroscopic technique for monitoring osteogenesis. This technique involves application of a radiofrequency (RF) pulse to excite a tissue or specimen at resonance, and the acquisition of signal in the form of RF energy during subsequent relaxation of tissue magnetization. This method provides quantitative parameters that are directly dependent on the tissue properties, such as the spin-spin T₂ relaxation time, which describes the time it takes the transverse component, to relax back to its equilibrium condition following excitation. MRM and related technologies are particularly useful as they are non-invasive and replace the conventional, invasive histological assessment of engineered bone tissue. Such histological assessment is particularly problematic in that it destroys material that may be otherwise suitable for implantation. In an aspect, the magnetic resonance spectroscopy provides a capability to assess implant stiffness or mineralization by calculating T₂ values. Generally, lower T₂ values are indicative of increased stiffness or mineralization.

Where the implant preferably has a spatial geometry, the cell population is introduced to a biocompatible scaffold or a sponge. The biocompatible scaffold is any material to which at least a portion of the cell population may attach and that does not adversely impact the cell population or generate a deleterious immune response after implantation. In an aspect, the biocompatible scaffold comprises an extracellular matrix protein, polyethylene glycol (PEG) scaffold, Poly-DL-lactic-co-glycolic acid (PLGA), gelatin sponges, agar, or collagen.

In another embodiment, the method accelerates osteogenesis by providing an isolated cell population capable of osteogenesis and exposing the cell population to a sonic or ultrasonic stimulation to accelerate osteogenesis of the cell population. The cell population is optionally a bone cell obtained from mesenchymal stem cells exposed to a bone cell-differentiating signal. The stimulation exposure can be on the cells outside the body (in vitro), on the cells that have been implanted back in the body (ex vivo), or both. The cells are optionally introduced to a biocompatible scaffold. The stimulation exposure is optionally an ultrasonic stimulation applied in a periodic manner. A periodicity of about 24 hours is convenient for ex vivo stimulation methods. In an embodiment, the stimulation is applied at least 5 times or more, between 10 and 30, or about 15 times over the course of three weeks or less.

In another embodiment, the invention is a method of implanting a cell or tissue implant into a patient in need of treatment by any of the methods disclosed herein. In this embodiment, the implantation is optionally an autologous implantation, where the cell population is obtained from the patient. To minimize tissue disruption, a useful cell population is obtained by removing mesenchymal stem cells from a patient's bone marrow. A particularly useful embodiment involves using the methods disclosed herein for generating implants suitable for repairing a bone tissue defect, wherein mesenchymal stem cells are exposed to a differentiating signal to generate bone cells such as osteoblasts, which are ultrasonically stimulated. The cells can be stimulated prior to or after introduction to scaffold suitable for bone implantation. In an aspect the patient is an animal, a mammal, or a human.

### BRIEF DESCRIPTION OF THE DRAWINGS

FIG. 1 (from Stock and Vacanti, Annu. Rev. Med. 52:443-51, 2001) is an example of a current process of bone tissue engineering where mesenchymal stem cells (MSCs) obtained from a patient are seeded on a scaffold, supplemented with growth factor to differentiate the cells into osteoblasts and cultured. After a substantial length of culture time, T_{C}, the scaffold with seeded cells is implanted into the patient, thereby restoring bone.

FIG. 2 is a flow-chart summary of one embodiment of the invention for reducing the culture time required before implanting the seeded scaffold.

FIG. 3 is a schematic illustration of ultrasound (US) treatment of osteoblast-differentiated cells seeded on a construct (OB Diff Construct) and cultured in a differentiation media (Diff Media).

FIG. 4 is T₂-weighted magnetic resonance (MR) images of OB Diff constructs acquired for: A. week 0; B. week 1; and C. week 2.

FIG. 5 is a plot of T₂ relaxation time (ms) as a function of time (weeks) after cells are introduced to a scaffold. OB Diff refers to osteoblasts; No US refers to no ultrasound treatment; US refers to ultrasound treatment; CON are control cells that are cultured in basal media and not exposed to ultrasonic stimulation.

FIG. 6 is a pixel by pixel mono-exponential T₂ map for the same constructs shown in FIG. 4: A. week 0; B. week 1; and C. week 2.

FIG. 7 shows H&E staining results at week 2 for CON (a), OB Diff No US (b), and OB Diff US (c) constructs. Image magnification is 20×.

FIG. 8 shows von Kossa staining results at week 2 for CON (a), OB Diff No US (b), and OB Diff US (c) constructs. Image magnification is 20×.

FIG. 9 shows OCN staining results at week 2 for CON (a), OB Diff No US (b), and OB Diff US (c) constructs. Image magnification is 20×.

### DETAILED DESCRIPTION OF THE INVENTION

“Ultrasound” or “ultrasonic” generally refers to sound waves having a frequency greater than the upper limit of human hearing, such as greater than about 20 kHz. “Sonic” refers to lower frequency wavelengths that are audible to human hearing, such as less than about 20 kHz, or between about 20 Hz and 20 kHz. “Sonic stimulation” or “ultrasonic stimulation” refers to an applied ultrasound or sound wave resulting in a cellular response by a cell population in such a manner so as to enhance an implant outcome parameter. A cell population can be “exposed” to this stimulation outside the patient (“in vitro”) and/or after it has been implanted into a patient (“ex vivo”) by means known in the art.

A patient in need of treatment refers to an individual who could benefit from an implant. For example, an individual suffering from a structural or functional bone defect could benefit from a tissue-engineered bone implant procedure of the present invention. Similarly, a patient suffering a cartilage defect, can benefit from a tissue-engineered cartilage implant procedure of the present invention.

“Implant outcome parameter” refers to a measurable or quantifiable effect by sonic or ultrasonic stimulation that improves implant effectiveness. For example, increasing the growth and proliferation of a cell population is an implant outcome parameter that reduces the time required to generate an implant material suitable for implantation in a patient. Such a time reduction results in an “enhanced” implant outcome parameter. In the example of a cell population comprising a bone cell, examples of implant outcome parameters include increased mineral deposition, increased osteogenesis. Other examples include increased extracellular matrix deposition, increased release of bioactive factors generated by cells in the implant, enhanced signal transduction or mechanotransduction.

“Cell population” is used broadly to refer to the cells that are to be implanted in the patient. In an embodiment, the cell population is a substantially homogeneous, or an isolated and purified, cell line. Alternatively, the cell population may have two or more distinct cell-type subpopulations. Cell population may be a type of stem cell capable of differentiating into a particular cell-type, such as a bone cell (e.g., osteoblast), cartilage cell (e.g., chondrocyte) or others. Cell population also refers to a tissue-specific cell type such as a bone cell, cartilage cell, etc. A “transplantable” cell population refers to cells that are useful for inserting into the body as an implant, such as bone cells or cartilage cells, for example. An implant optionally contains a biocompatible scaffold.

A preferred cell population comprises a cell type that is an osteoprogenitor cell, such as an osteoprogenitor cell located in the periosteum and/or the bone marrow. An osteoprogenitor cell is capable of differentiating in response to a signal into a cell responsible for bone formation, such as an osteoblast. For example, mesenchymal stem cells are capable of differentiating into osteoblasts when cultured in a media containing growth factors, such as bone morphogenetic proteins. Whether a cell population has sufficiently differentiated into cells responsible for bone formation can be assessed by measuring or monitoring any one or more bone markers known in the art, or by magnetic resonance spectroscopic techniques disclosed herein.

A “cell or tissue implant” refers to material that is suitable for insertion into a patient in need of treatment and the term encompasses individual cells that are relatively unorganized (e.g., not anchored to a substrate) or an organized set of cells and extracellular matrix that is anchored or connected to a substrate or scaffold. Depending on the treatment required, the implant can comprise cells suspended in a liquid medium in which the cell suspension is injected into the patient, or cells attached to a scaffold in which the scaffold plus cells are implanted into the patient.

“Expanding” a cell population refers to culturing the cells in a manner so that cell number increases (e.g., the cells proliferate). This expansion step can be useful if there is a limited number of starting cells, and the implant requires a significantly larger number of cells.

The sonic or ultrasonic stimulation is optionally described in terms of one or more physical variables. “Intensity” is expressed in terms of power per unit area. “Low intensity” refers to a stimulation intensity that does not generate significant tissue or cellular damage, including damage such as by heating. For example, higher-intensity ultrasound (100 mW/cm²-770 mW/cm²) has been shown to produce heat. “Low intensity” refers to about 60 mW/cm² or less. In an aspect, any of the methods disclosed herein relate to cellular stimulation by low-intensity ultrasound.

“Operating frequency” refers to the frequency of the sonic or ultrasonic signal, and can range from 20 Hz to 20 kHz for sonic stimulation to above 20 kHz for ultrasonic stimulation. “Pulse width” refers to the length of time for which an individual ultrasonic signal is generated. The pulse width itself is repeated at a “pulse repetition rate”. “Daily treatment duration” or “individual treatment duration” refers to the length of time for which the stimulation is applied. In an aspect, the treatment occurs daily. Overall treatment duration is determined by the time between daily or individual treatments and the number of times the daily or individual treatments are applied.

“Scaffold” refers to a material upon which cells can attach in a two- or three-dimensional configuration. The term encompasses artificial constructs known in the art as well a basic homogeneous composition to which cells can attach. In an aspect the scaffold itself is biocompatible, or coated with a material that makes the scaffold biocompatible. “Biocompatible” refers to a material that does not cause a deleterious immune response resulting in implant rejection. A cell population is “introduced” to a scaffold by, for example, immersing the scaffold in a solution of cells, or applying a cell-containing solution to a scaffold.

“Osteogenesis” refers to bone development, and more specifically the process by which new bone is generated by bone cells such as osteoblasts. A cell population capable of osteogenesis includes bone cells such as osteoblasts and osteoclasts and may include related cells from mesenchymal stem cell differentiation such as chondrocytes and adipocytes.

In vitro “ultrasonic exposure” refers to ultrasonic stimulation that occurs outside the patient. Ex vivo “ultrasonic exposure” refers to ultrasonic stimulation of cells that were once removed from the body, but have been subsequently implanted in the patient.

All references cited throughout this application, for example patent documents including issued or granted patents or equivalents; patent application publications; and non-patent literature documents or other source material are hereby incorporated by reference in their entireties, as though individually incorporated by reference, to the extent each reference is not inconsistent with the disclosure in this application (for example, a reference that is partially inconsistent is incorporated by reference except for the partially inconsistent portion of the reference).

Every formulation or combination of components described or exemplified herein can be used to practice the invention, unless otherwise stated. All patents and publications mentioned in the specification are indicative of the levels of skill of those skilled in the art to which the invention pertains. References cited herein are incorporated by reference in their entirety to indicate the state of the art as of their publication or filing date and it is intended that this information can be employed herein, if needed, to exclude specific embodiments that are in the prior art. Whenever a range is given in the specification, for example, a time range, frequency range, intensity range, or a composition range, all intermediate ranges and subranges, as well as all individual values included in the ranges given are intended to be included in the disclosure

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein may be practiced in the absence of any element or elements, limitation or limitations which is not specifically disclosed herein.

One of ordinary skill in the art will appreciate that starting materials, materials, reagents, synthetic methods, purification methods, analytical methods, assay methods, and methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

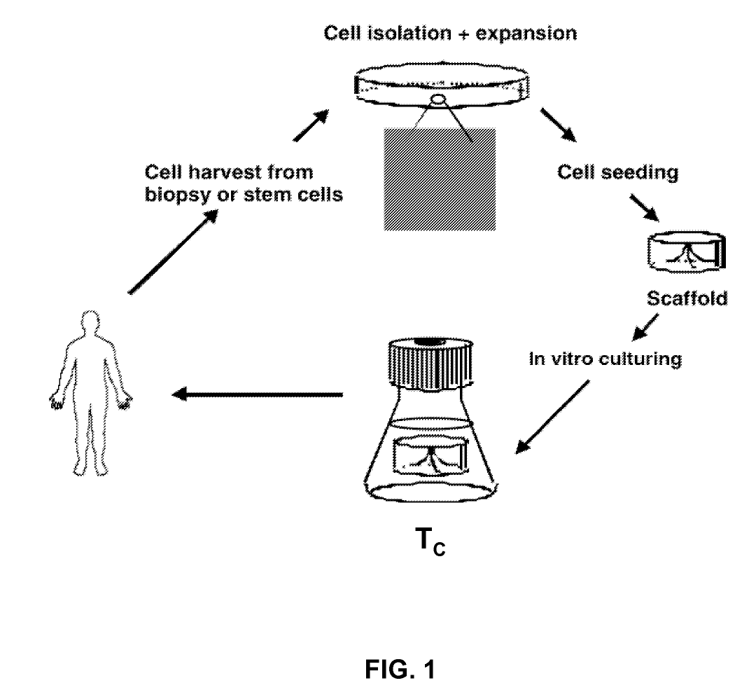

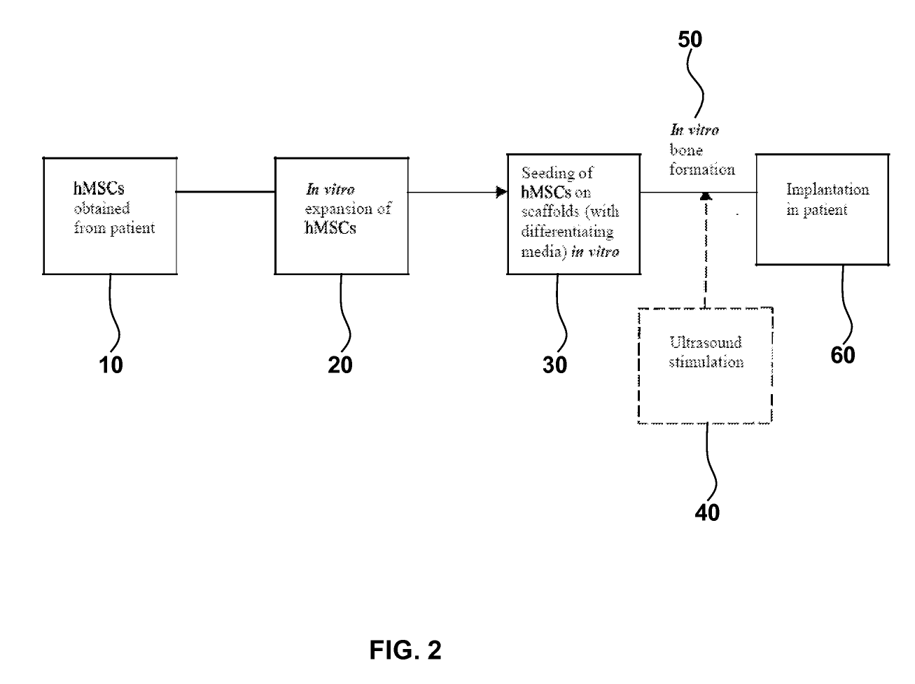

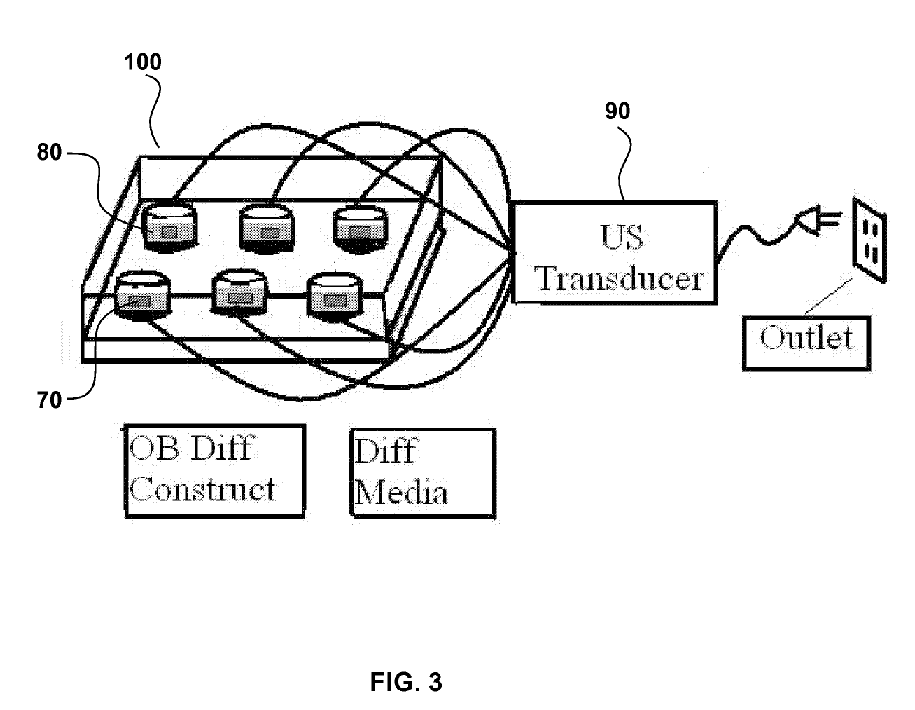

An exemplified embodiment is a method of providing a bone implant from human mesenchymal stem cells (hMSCs) using ultrasonic stimulation. FIG. 2 summarizes the steps involved in that method. Referring to FIG. 2, hMSCs are obtained from a patient 10, and are expanded 20. After a sufficient time, the hMSCs are seeded or introduced to a scaffold 30 in vitro. Differentiating media is provided to the hMSCs so that they differentiate into bone-producing cells (e.g., osteoblasts). The seeded scaffold is subjected to ultrasound stimulation 40 and in vitro bone formation 50 monitored against a control seeded scaffold that has not been stimulated with ultrasound. After the seeded scaffold is ready, it is implanted into the patient 60. Methods disclosed herein use US-stimulation to increase the rate of cell growth and proliferation. In an aspect, referring to FIG. 1, US application significantly reduces T_{C}, such as by at least 20%, 25, or 50%. FIG. 3 provides an example of a setup 100 for a set-up to simultaneously expose multiple scaffolds 70 seeded with hMSCs and bathed within a differentiating media 80 to ultrasound by an ultrasound transducer 90. In other aspects of the invention, steps 20 and 30 are not performed, and instead cell population obtained in step 10 are exposed to US stimulation 40, and the implantation in the patient involves a US-stimulated cells only.

Ultrasound Accelerated Bone Tissue Engineering Monitored with Magnetic Resonance Microscopy:

Tissue engineering has the potential to treat bone loss, but current bone restoration methods, including osteogenesis from mesenchymal stem cells (MSCs), require three to four weeks for bone formation to occur. In this study, we stimulate the formation of engineered bone tissue with low-intensity ultrasound, which has been proven to accelerate bone healing in vivo. One group of engineered bone constructs receives ultrasound stimulation 20 minutes per day over a 3-week growth period. We monitor the growth of all the engineered constructs quantitatively and noninvasively using magnetic resonance microscopy (MRM), where the T₂ relaxation times of all the constructs are measured, on a weekly basis, using an 11.74 T Bruker spectrometer. Histological and immunocytochemical sections are obtained for all constructs and correlated with the MR results. This study shows that ultrasound accelerates osteogenesis in vitro for tissue engineered bone, the growth and development of which can be monitored using MRM.

Millions of patients experience bone loss as a result of degenerative disease, trauma, or surgery [1]. According to Wolff's “Law of Bone Remodeling”, changes occur in the bone architecture allowing restoration of its normal function to meet the mechanical demands imposed on it [2]. However, this capacity is limited when there is insufficient blood supply, mechanical instability, or competition with highly proliferating tissues [3].

The current “gold standard” for specific-site, structural and functional bone defect repair is autologous bone grafts. However, this solution presents certain complications such as donor site morbidity, infection, malformation, and subsequent loss of graft function. Another widely employed technique is transplantation of allograft bone, which presents the risks of potential disease transmission and host rejection, and suffers from limited supply [4].

Implants developed via tissue engineering may be a more viable solution to the problem of bone loss, since biocompatibility will no longer be an issue, and the implants will be more readily available to patients [5].

One bone tissue engineering strategy currently employed is illustrated in FIG. 1. After cellular expansion of mesenchymal stem cells (MSCs) obtained from the patient, these are seeded on biodegradable and biocompatible scaffolds [6], and supplemented with growth factors that enable them to differentiate into osteoblasts (bone-forming cells) [7]. After a substantial culture period (T_{C}), the scaffold is implanted into the patient, leading to bone restoration [8].

Reducing the culture time (T_{C}) of stem cells is necessary to increase the effectiveness of the implants. The use of electrical and mechanical stimulation to accelerate stem cell differentiation has been implemented, but the optimized usage of such techniques has yet to occur [9]. In this study, we stimulated the growth of tissue engineered bone constructs with US (see FIG. 2), thereby effectively reducing T_{C} by as much as about 50%. The reasons behind exploring US stimulation are: (a) US waves are noninvasive, which is very relevant to this study to insure the integrity of the constructs; (b) previous studies showed that low-intensity pulsed US, administered with a dose as short as 20 minutes per day, activated ossification in vitro via a direct effect on osteoblasts and ossifying cartilage, after other animal and clinical studies showed that low-intensity US accelerated bone healing in vivo [10].

Magnetic resonance imaging is widely used in vivo to assess connective tissue degeneration [11]. It has also been effective in studying ectopic bone formation in the rat in vivo [12]. MRM has been used to investigate the regeneration of engineered tissue [13]. A recent study showed that MRM can be used to monitor osteogenesis in tissue-engineered constructs [8]. In this work, we study the feasibility of using US stimulation to enhance and hasten osteogenesis in tissue engineered constructs. The periodic monitoring of tissues T₂ relaxation time correlated with histological and immunocytochemical analyses is used to further assess the results.

Specimen Preparation: Mesenchymal stem cells (MSCs) isolated from fresh adult human bone marrow is provided by AllCells® (AllCells LLC, Berkeley, Calif.). Nucleated cells are expanded via incubation for 3 weeks in a basic culture medium composed of Dulbecco's Modified Eagle's Medium supplemented with 10% fetal bovine serum and 1% antibiotics at 37° C. Helistat® absorbable collagen scaffolds (Integra LifeSciences Corporation, Plainsboro, N.J., USA) are trimmed into 3 mm×3 mm×5 mm pieces for use as the biological scaffold. Tissue engineered constructs are generated by seeding the collagen scaffolds with MSCs at a density of 2×10⁶ cells/ml with a slight vacuum created with a 20 ml syringe. The mixture of scaffolds and cell suspensions is incubated at 37° C. for 2 hours. Then, the cell-seeded constructs are divided into 3 groups: control (CON) constructs are cultured in basal media (same composition as basic culture medium described above), differentiated, non-stimulated (OB Diff No US) constructs and differentiated, ultrasound stimulated (OB Diff US) constructs are cultured in differentiating media (basal media with 100 mM dexamethasone, 100 mM β-glycerophosphate, and 50 mg/ml ascorbic acid-2-phosphate, which are factors promoting the osteogenic differentiation of human MSCs). The 3 groups are allowed to grow in vitro for 3 weeks.

Ultrasound Stimulation Treatment: The US treatment is administered in a therapy unit consisting of a sonic accelerated fracture-healing system (SAFHS) device (model 2A; EXOGEN, Memphis, Tenn., USA) and 6 transducers (with coupling gel), connected to a multiwell plate filled with 6 ml of tissue culture medium, and the engineered constructs, OB Diff US (FIG. 3). The transducers delivered pulsed US waves with an intensity of 30 mW/cm², operating frequency of 1.5 MHz, pulse width of 200 μsec and pulse repetition rate of 1 kHz. The duration of each treatment is 20 minutes per day through the growth period.

Magnetic Resonance Imaging System: MRI experiments are conducted at 11.74 T (500 MHz for protons) using a 56 mm vertical bore magnet (Oxford Instruments, Oxford, UK) and a Bruker DRX Avance Spectrometer (Bruker Instruments, Billerica, Mass. USA) controlled by a Silicon Graphics SGI2 workstation (Mountain View, Calif., USA). MR images are acquired using a Bruker Micro 5 imaging probe with triple axis gradients (maximum strength 2 T/m), and a 10 mm diameter RF saddle coil is used to transmit/receive the nuclear magnetic resonance signals.

MRI and Measurements of MR parameters: OB Diff No US, OB Diff US, and CON constructs (n=2) are studied at 4 growth stages, referred to as weeks 0, 1, 2, and 3. Axial slices are taken along the axis of the test tube and positioned at the center of each specimen. For each sample, the spin-spin relaxation time (T₂) is measured and averaged for specific regions of interest (ROIs) localized at the periphery of each construct. T₂ was measured using a spin echo imaging pulse sequence with 32 echoes (repetition time TR=4 s, echo time TE=7 ms, NEX=1, matrix dimensions=128×128).

The T₂ values are calculated using laboratory built software in MATLAB 7.0 (MathWorks INC., Natick, Mass.), which performs least square fitting of the experimental MR data for each ROI to calculate the mono-exponential T₂ relaxation time (1).
SNR(TE)=SNR₀e^{-TE/T}₂ (1)
where SNR(TE) is the SNR value at a specific TE value, and SNR₀ is the initial SNR value.

In addition, pixel by pixel T₂ mapping is produced for all the imaged constructs using a laboratory built MATLAB software, in order to maximize the contrast information throughout the construct.

Histological and Immunocytochemical Analyses: Following MR testing, one set of tissue engineered constructs containing the 3 different experimental groups is washed with phosphate buffered saline (pH 7.4), then fixed in 10% formal in, every week throughout the growth period. All fixed samples are sent to Histoserv, INC. (Germantown, Md. USA), sectioned at 5 μm thickness, and stained for histological and immunocytochemical analyses. Hematoxylin and Eosin (H&E) staining was performed to detect cell nuclei in purple, over the collagen matrix, in pink. This provides information related to the increase of cell proliferation with time. Also, von Kossa staining is performed to examine the mineralization during osteogenic differentiation due to calcium deposition. The tissue sections are treated with a sliver nitrate solution and the silver is deposited by replacing the calcium [8]. Furthermore, staining for osteocalcin (OCN), a bone matrix protein, is performed to ascertain the presence of bone tissue in the differentiated constructs. In this stain, the copper regions are a mark of OCN presence.

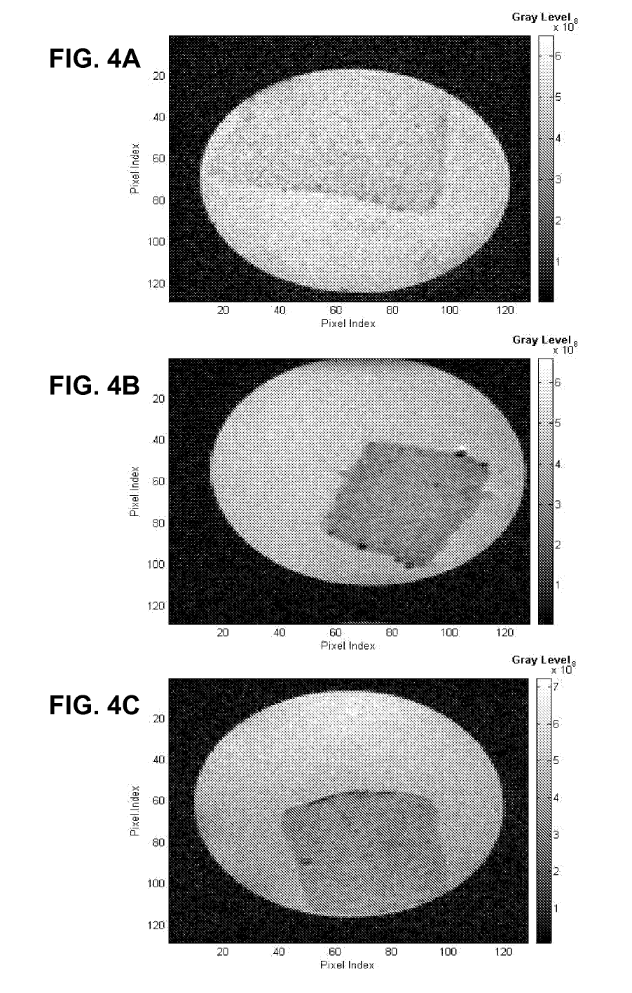

Dependence of MR Parameters on Engineered Bone Tissue Formation and Comparison of US Stimulated Constructs with Non-Stimulated Ones: T₂-weighted MR magnitude axial images of US stimulated osteogenic constructs at weeks 0, 1, and 2 are shown in FIG. 4. Spin-echo imaging pulse sequence is used with TR=4000 ms, TE=140 ms, slice thickness=0.5 mm, and matrix dimensions=128×128. The intensity of the MR images for the osteogenic constructs decreased with tissue development.

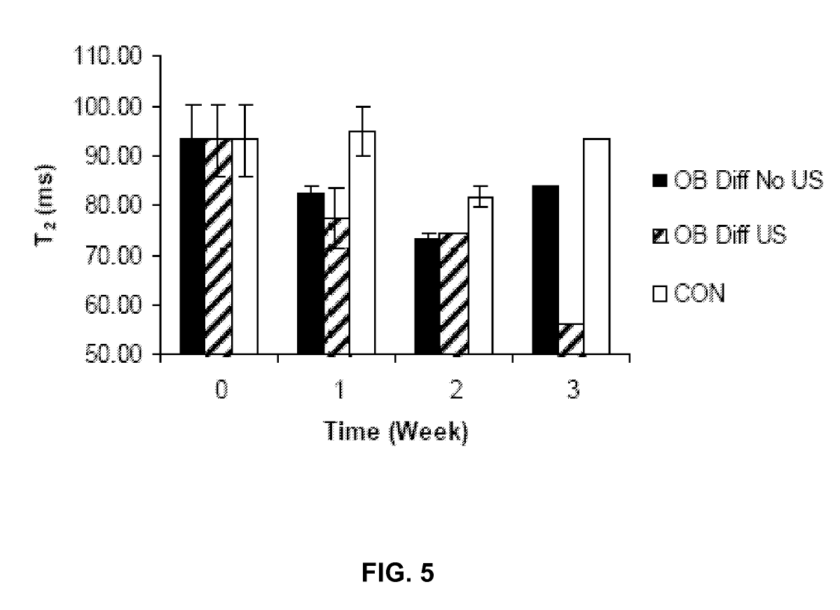

FIG. 5 compares the variation of T₂ relaxation time calculated weekly, using mono-exponential fitting, for peripheral ROIs in the constructs, for each of the 3 different experimental groups, over the 3-week developmental period. The T₂ relaxation time decreases with time for the osteogenic constructs (OB Diff No US and OB Diff US), whereas that for the control constructs (CON) does not show any similar decrease. Starting with initial values of 96.6 ms and 97.8 ms at week 0, the T₂ relaxation time reaches a value as low as 56.0 ms for the US stimulated osteogenic constructs, whereas it only reaches values of 72.1 ms and 74.1 ms for the non-stimulated osteogenic constructs; the lowest T₂ values calculated for the control group are 80.2 ms and 83.1 ms.

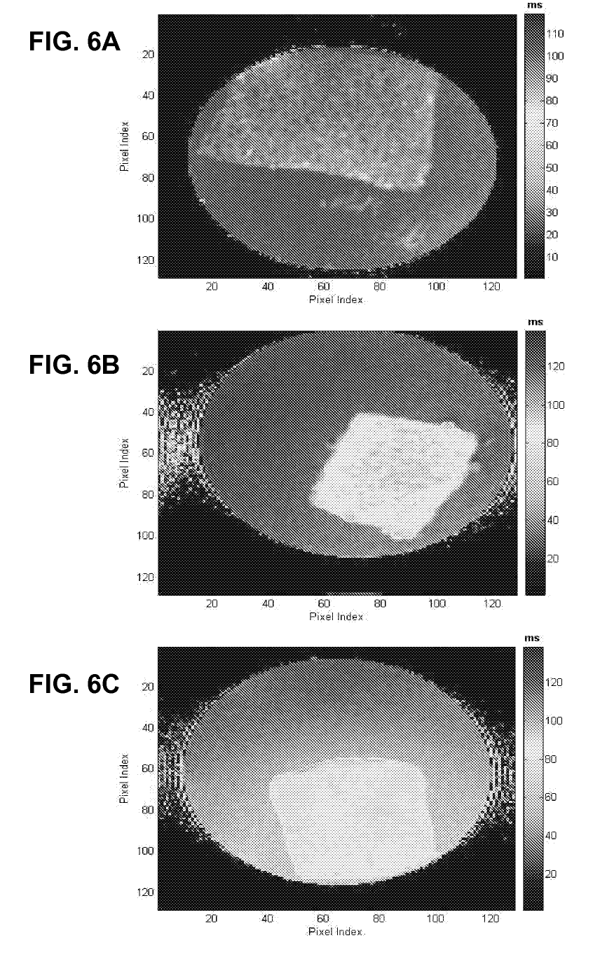

FIG. 6 displays pixel by pixel T2 relaxation time maps of the same samples shown in FIG. 4. The vertical legend bar to the right of each map shows the T₂ values in ms that each color shade represents. The variation of the dominant color from red (FIG. 6A) (value about 100-120), yellow (FIG. 6B) (about 80), to green-blue (FIG. 6C) (about 20-60), in the region of the map occupied by the construct, demonstrates a decrease in the computed T₂ values when going from week 0 (FIG. 6A: T₂ about 100 ms) to week 2 (FIG. 6C: T₂ about 60 ms). This effect is actually present for both osteogenic construct groups, as shown in FIG. 5. It can be noticed by examining those maps that they contain better contrast information than what is displayed in the T₂-weighted MR magnitude images shown in FIG. 4, and therefore, provide better tissue characterization throughout the construct region.

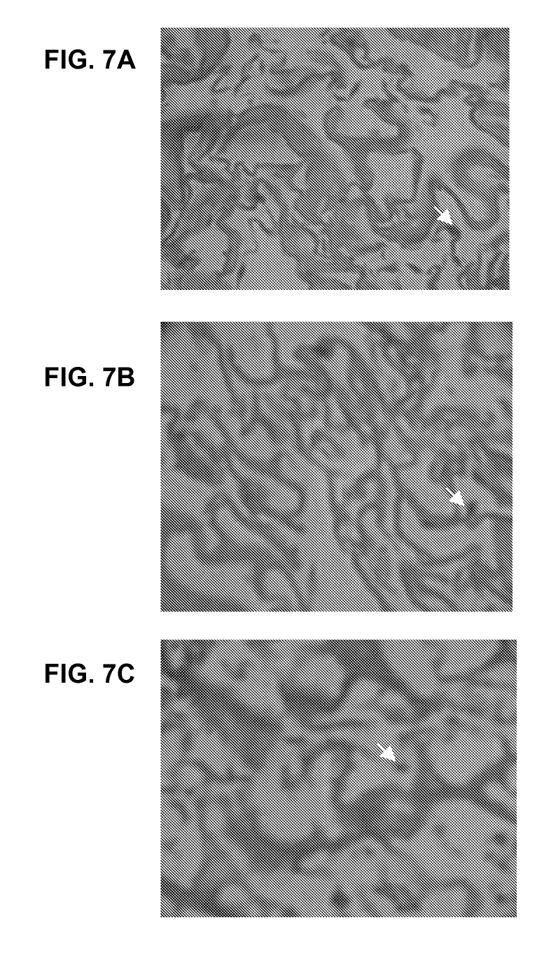

FIG. 7 shows the H&E staining results at week 2 for the 3 construct groups: A. CON (control); B.; OB Diff No US C. OB Diff US. In all constructs, collagen is stained by a lighter shade, and cell nuclei are dark. A white arrow points to a nucleus in each of the panels A-C. The US stimulated osteogenic construct has more cells than the non-stimulated osteogenic construct at week 2, indicating greater cell proliferation in the construct receiving ultrasound treatment.

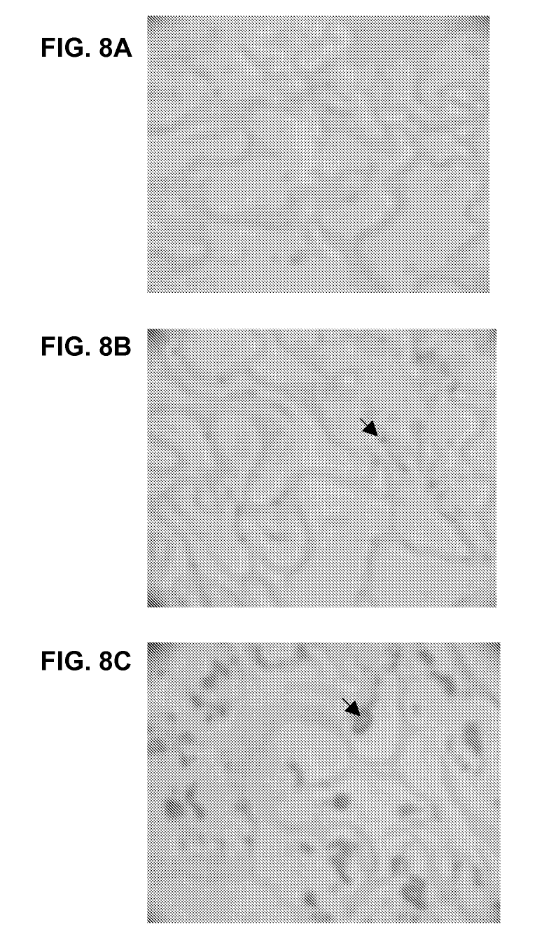

FIG. 8 shows the von Kossa staining results at week 2 for the 3 construct groups. A. CON (control); B.; OB Diff No US C. OB Diff US. Black nodules indicate calcium deposition, which increases with osteoblast formation; the black arrow points to one nodule. Black nodules are absent from the control constructs, but apparent in the osteogenic ones (US stimulated and non-stimulated) at week 2, indicating calcium deposition in these only. The US stimulated construct, as shown, has more black nodules than the non-stimulated one, indicating greater calcium deposition in the construct receiving US treatment.

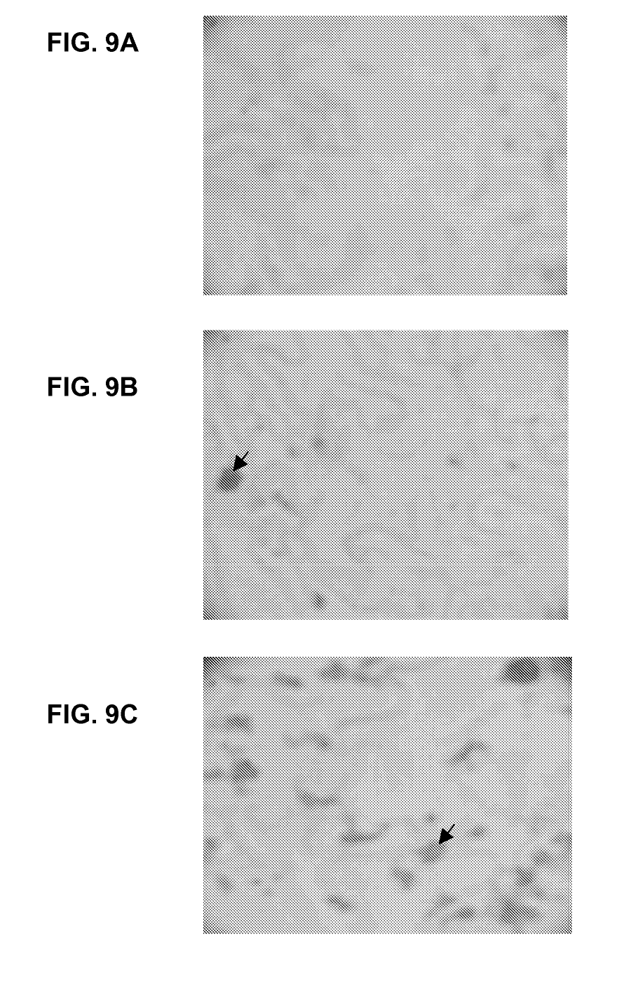

FIG. 9 shows the results of staining for OCN at week 2 for the 3 construct groups. A. CON (control); B.; OB Diff No US C. OB Diff US. The lighter-colored copper regions indicate the presence of OCN. One of the copper colored regions is indicated by the black arrow. Copper colored regions are absent from the control constructs, but apparent in both osteogenic constructs, indicating the presence of OCN in these only. The US stimulated construct has more copper colored regions than the non-stimulated osteogenic one, indicating greater presence of osteocalcin in the construct receiving US treatment.

Both von Kossa and OCN stains show further mineralization and bone formation in the US stimulated osteogenic constructs than in the non-stimulated ones, while having a faster decrease in T₂ values measured for the US stimulated constructs. Therefore, there is good correlation between the histological/immunocytochemical and the MR results, implying that the faster decrease in T₂ values over the growth period can be directly correlated to the faster increase in stiffness, due to improved mineral deposition.

Both von Kossa and OCN stains show further mineralization and bone formation in the US-stimulated osteogenic constructs than in the non-stimulated ones, while having a faster decrease in T₂ values measured for the US stimulated constructs. Therefore, there is good correlation between the histological/immunocytochemical and the MR results, implying that the faster decrease in T₂ values over the growth period can be directly correlated to the faster increase in stiffness, due to improved mineral deposition.

This work is unique in two aspects: First, it shows that MRM is sensitive enough to characterize the acceleration of osteogenic constructs growth, by sensing the difference in T₂ values between the US stimulated and non-stimulated constructs. Second, it shows that US is effective in accelerating osteogenesis in vitro. The decrease in the T₂ values over time for the osteogenic constructs can be explained by the introduced magnetic susceptibility upon mineral deposition [8], and translated into an increase in stiffness of the tissue.

Furthermore, a decrease in the size of the osteogenic constructs over the incubation period Is observed, and Is more evident for the US stimulated constructs than the nonstimulated ones. This may be due to normal growth consolidation or non-uniform cell seeding in the collagen scaffold [8]. However, the degradation of collagen scaffolds by osteoclasts along with the formation of new bone matrix by osteoblasts has been proven in a recent study [14]. Therefore, the MSCs possibly differentiating into both of these bone cell types allow degradation of the collagen scaffold matrix upon mineralization. This fact is most likely the major reason for the decrease in size observed in the osteogenic constructs, and offers an advantage for bone remodeling using tissue engineered constructs, where biodegradability of the scaffold material is very important [14]. In addition, we noticed a better decrease in T₂ values over time in the peripheral than in the central parts of the constructs, which means that more mineralization occurs at the periphery of the constructs where the highest cell density probably resides. This problem may be overcome by improving the cell seeding procedure.

### REFERENCES

J. A. Buckwalter, and T. D. Brown, “Joint injury, repair, and remodeling: roles in post-traumatic osteoarthritis,” Clin. Orthop. Relat. Res., vol. 423, pp. 7-16, 2004.
J. Wolff, The Law of Bone Remodelling. Germany: Springer-Verlag Berlin Heidelberg, 1986.
A. L. Pinheiro, and M. E. Gerbei, “Photoengineering of bone repair processes,” Photomedicine and Laser Surgery, vol. 24, issue 2, pp. 169-178, 2006.
J. R. Mauney, V. Volloch, and D. L. Kaplan, “Role of adult mesenchymal stem cells in bone tissue-engineering applications: current status and future prospects,” Tissue Engineering, vol. 11, issue 5/6, pp. 787-802, 2005.
J. R. Jones, L. M. Ehrenfried, L. L. and Hench, “Optimizing bioactive glass scaffolds for bone tissue engineering,” Biomaterials, vol. 27, issue 7, pp. 964-973, March, 2006 (Article in press).
U. A. Stock, and J. P. Vacanti, “Tissue Engineering: Current State and Prospects,” Annu. Rev. Med., vol. 52, pp. 443-451, 2001.
H. Kim, H. Suh, S. A. Jo, H. W. Kim, J. M. Lee, E. H. Kim, Y. Reinwald, S. H. Park, B. H. Min, and I. Jo, “In vivo bone formation by human marrow stromal cells in biodegradable scaffolds that release dexamethasone and ascorbate-2-phosphate,” Biochemical and Biophysical Research Communications, vol. 332, pp. 1053-1060, May, 2005.
H. Xu , S. F. Othman, L. Hong, I. A. Peptan, and R. L. Magin, “Magnetic resonance microscopy for monitoring osteogenesis in tissue-engineered construct in vitro,” Magnetic Resonance in Medicine, vol. 54, pp. 605-615, 2005 (Article in Press).
S. H. Cartmell, J. Magnay, A. El Haj, and J. Dobson, “Use of mechanical particles to apply mechanical forces for bone tissue engineering purposes,” Journal of Physics: Conference Series, vol. 17, pp. 77-80, 2005.
P. A. Nolte, J. Klein-Nulend, G. H. Albers, R. K. Marti, C. M. Semeins, S. W. Goei, and E. H. Burger, “Low-intensity ultrasound stimulates endochondral ossification in vitro,” Journal of Orthopaedic Research, vol. 19, pp. 301-307, 2001.
D. D. Stark, and W. G. Bradley, Magnetic Resonance Imaging. St. Louis, Mo.: C. V. Mosby, 3rd edition, 1999.
E. H. M. Hartman, J. A. Pikkemaat, J. W. M. Vehof, A. Heerschap, J. A. Jansen, and P. H. M. Spauwen, “In vivo magnetic resonance imaging explorative study of ectopic bone formation in the rat,” Tissue Engineering, vol. 8, issue 6, pp. 1029-1036, 2002.
I. Constantinidis, and A. Sambanis, “Noninvasive monitoring of tissue-engineered constructs by nuclear magnetic resonance methodologies,” Tissue Engineering, vol. 4, pp. 9-17, 1998.
H. Domaschke, M. Gelinsky, B. Burmeister, R. Fleig, T. Hanke, A. Reinstorf, W. Pompe, and A. Rosen-Wolff, “In vitro ossification and remodeling of mineralized collagen I scaffolds,” Tissue Engineering, vol. 12, issue 4, pp. 949-958, 2006.
Aaron R. K., et al. (2004). “Treatment of non-unions with electric and electromagnetic fields”, Clinical Orthopaedics, 419, 21-29.
Lakes, R. (2005). “Electrical properties of bone, a review”. Available at http://silver.neep.wisc.edu/˜lakes/BoneElectr.html. Accessed Nov. 30, 2005.
Moinnes, J. J., Vidula, N., Halim, N., & Othman, S. F. (2006). “Ultrasound Accelerated Bone Tissue Engineering Monitored with Magnetic Resonance Microscopy.” Proceedings of the 28^{th} Annual International Conference of the IEEE Engineering in Medicine and Biology Society.
Nishimura, D. G. (1996). Principles of Magnetic Resonance Imaging. Department of Electrical Engineering, Stanford University.
Nordin, M. & Frankel, V. (2001). Basic Biomechanics of the Musculoskeletal System, Philadelphia: Lippincott Williams & Wilkins.
Pilla, A. A. (2002). “Low-intensity electromagnetic and mechanical modulation of bone growth and repair: are they equivalent?”, Journal of Orthopaedic Scienc., 7, 420-428.
Rahaman, M. N. & Mao, J. J. (2005). “Stem cell based composite tissue constructs for regenerative medicine.” Biotechnology Bioengineering, 91(3), 261-284.
Wu, Y. et al. (2006). “Bone tissue engineering evaluation based on rat calvaria stromal cells cultured on modified PLGA scaffolds.” Biomaterials, 27(6), 896-904.
Zhang, Y., Cheng, X., et al. (2006). “Novel chitosan/collagen scaffold containing transforming growth factor-betal DNA for periodontal tissue engineering.” Biochem Biophys Res Commun., 344(1), 362-369.

We claim:

1. A method of preparing a cell or tissue implant for insertion into a patient in need of treatment, said method comprising:
obtaining a transplantable cell population;
culturing said cell population in a culture media; and
exposing said cell population to a sonic or ultrasonic stimulation, wherein said stimulation provides a capability for an enhanced implant outcome parameter.

2. The method of claim 1 further comprising:
providing a biocompatible scaffold; and
introducing said cell population to said scaffold.

3. The method of claim 2, wherein said stimulation occurs after said introducing step.

4. The method of claim 1, wherein said implant outcome parameter is selected from one or more of the group consisting of:
accelerated cell growth or proliferation;
reduced time for implant generation;
increased mineral deposition; and
increased osteogenesis.

5. The method of claim 1, wherein said cell population is obtained from said patient.

6. The method of claim 5 further comprising expanding said cell population prior to said introducing step.

7. The method of claim 6, wherein said introducing step comprises applying said cells having a concentration selected from a range of between 1×10⁶ to 1×10⁷ cells/mL to said biocompatible scaffold.

8. The method of claim 1, wherein said cell population is not obtained from said patient.

9. The method of claim 1, wherein said cell population comprises mesenchymal stem cells.

10. The method of claim 9, further comprising differentiating at least a portion of said mesenchymal stem cells into osteoblast cells prior, during, or prior and during said ultrasonic stimulation.

11. The method of claim 1, wherein said sonic or ultrasonic stimulation has:
an operating frequency of between 10 Hz and 10 MHz; and
a pulse repetition rate selected from between 500 Hz and 5 kHz

12. The method of claim 1, wherein said stimulation is a low-intensity ultrasonic stimulation having:
an intensity of 30 mW/cm²;
an operating frequency of 1.5 MHz;
a pulse width of 200 μsec; and
a pulse repetition rate of 1 kHz.

13. The method of claim 1, wherein the ultrasound is applied daily, with each daily treatment having a daily treatment duration selected from a range between 10 minutes and 30 minutes and wherein the daily treatment is repeated from a range between 5 days and 21 days.

14. The method of claim 13, wherein the daily treatment is about 20 minutes per day.

15. The method of claim 1, further comprising implanting said implant into a patient.

16. The method of claim 1, wherein said implant is a bone implant and said outcome parameter comprises accelerated osteogenesis resulting in a decreased time required for bone implant generation compared to a bone implant not exposed to said sonic or ultrasonic stimulation, wherein said time is decreased by about 25% or better relative to a bone implant not stimulated with said sonic or ultrasonic stimulation.

17. The method of claim 16 further comprising monitoring the ultrasonically-stimulated cell population by magnetic resonance microscopy to measure said osteogenesis.

18. The method of claim 1 wherein the biocompatible scaffold comprises an extracellular matrix protein, polyethylene glycol, agar, or collagen.

19. A method of accelerating osteogenesis comprising:
providing an isolated cell population capable of osteogenesis; and
exposing said cell population to a sonic or ultrasonic stimulation;
thereby accelerating osteogenesis of said cell population.

20. The method of claim 19, wherein said cell population comprises a bone cell obtained from mesenchymal stem cells exposed to a bone cell-differentiating signal.

21. The method of claim 19, wherein said sonic or ultrasonic exposure is in vitro, ex vivo, or both in vitro and ex vivo.

22. The method of claim 19 further comprising:
providing a biocompatible scaffold; and
introducing said cell population to said biocompatible scaffold.

23. The method of claim 22, wherein said exposure comprises an ultrasonic stimulation that is applied in a periodic manner, having a periodicity of about 24 hours, and repeated at least 5 times or more.

24. A method of implanting a cell or tissue implant into a patient in need of treatment comprising:
obtaining a transplantable cell population;
culturing said cell population in a culture media;
exposing said cell population to a sonic or ultrasonic stimulation, wherein said stimulation provides a capability for an enhanced implant outcome parameter; and
implanting said tissue implant having an enhanced implant outcome parameter to said patient.

25. The method of claim 24, further comprising:
providing a biocompatible scaffold; and
introducing said cell population to said scaffold.

26. The method of claim 24, wherein said transplantable cell population comprises mesenchymal stem cells.

27. The method of claim 26, wherein said cell population is obtained from said patient.

28. The method of claim 26, wherein at least a portion of said mesenchymal stem cells are exposed to a differentiating stimulation thereby generating differentiation of at least a portion of said mesenchymal stem cells to osteoblasts.
